(19) United States
(12) Patent Application Publication
(10) Pub. No.: US 2019/0224034 A1
(43) Pub. Date: Jul. 25, 2019

(54) Head Support

(71) Applicant: Matthias Mersmann, Lichtenbusch (BE)

(72) Inventor: Matthias Mersmann, Lichtenbusch (BE)

(21) Appl. No.: 16/313,699

(22) PCT Filed: Jun. 22, 2017
(86) PCT No.: PCT/DE2017/000180

§ 371 Date: Dec. 27, 2018

(30) Foreign Application Priority Data
Jun. 27, 2016 (DE) 10 2016 007 740.9

(51) Int. Cl. A61F 5/058; A61F 5/055

(52) U.S. Cl. CPC A61F 5/05883; A61F 5/3707; A61F 5/055

(57) ABSTRACT

The invention relates to a head support, which is formed as a one-piece symmetrical shaped geometrical body having substantially an X-shape with a central middle part, from which legs stretch outwardly each diagonally opposite to each other. The head support also includes a neck strap that holds the head support in place between the chin and upper chest area of the person wearing the head support. The head support is easy to handle and its X-shape offers several advantages. This includes the adaptation of the same to the body of the person on two different support heights.

The invention relates to a head support device for the support of a human head in upright position of the body. The support is generated in the direction of the chest of the person wearing the head support device whereas an upper part of the head support builds a chin support bearing for the chin and the opposite end of the chin support creates a chest support bearing which rests at the upper chest area of the person and the head support device comprises a neck strap which is led around the neck of the person using the head support device and which holds the head support device in position between the chin and the upper chest area of the person.

Such head support is already known from the German publication DE 10 2014 012 071 A1. This head support as developed by the applicant himself enables its user to adopt a comfortable and relaxed head position in upright body posture especially when seated upright. This is possible because the headrest allows the cervical muscles of the user to relax while the head support supports these muscles by taking over the weight of the head. The known head support is designed such that it comprises an upper end which acts as a chin support bearing for the chin of the user and a lower end which acts as a chest support bearing for the upper chest area of the user. This support device is held in position by a neck strap, which is led around the neck of the user. According to DE 10 2014 012 071 A1 the support device can consist of and be made from a linkage girder construction, namely from rods where soft pads can be arranged at the upper and lower ends of these rods. The parts coming into contact with the user's body can alternatively also be created as inflatable soft body parts. Additionally, it is mentioned that the complete support device may comprise a single inflatable body where the exact shape of this body is not described.

The aforementioned known head support device however has the disadvantage that its embodiment does not take into account the fact that the users of such head support devices are of different body size and have different body proportions especially with respect to the size and shape of their chins as well as to the distance between their chins and upper chest areas. Additionally, the described head support allows only one head tilt angle which means that the head can be held in only one angle with respect to the spinal column. This can be disadvantageous in certain occasions, for instance when the person is sitting in a seat, for example in an airplane seat, the backrest of which is not oriented completely upright.

Therefore, the present invention is based on the objective to further develop the head support such that these inherent disadvantages are overcome and furthermore a head support is created which allows the user to adopt different head tilt angles and which is more suitable to be used by users with different body sizes and different distances between their chin and their upper chest areas. A further function of the head support shall also be to avoid lateral tilting of the head. Finally, the new head support shall be easy to handle, easy to manufacture as well as ergonomically advantageous.

This objective is solved with a head rest of this generic type by its embodiment as a single-piece symmetrical shaped geometrical body such that this body is essentially created as an X-shaped embodiment in a way that it comprises a central middle-piece and four legs which extend outward each diagonally opposite to each other, where two adjacent legs together with their connecting concave outer face of the middle-piece form the chin support bearing and the two opposite legs together with their connecting concave outer face of the middle-piece forms the chest support bearing, where the outer faces of the middle-piece connecting each one leg of the chin support bearing and one leg of the chest support bearing are also shaped concavely and where at the intersection of the diagonals from the four legs at the outer side of the middle-piece, which is facing away from the neck of the user, there are means to connect the neck strap.

The solution according to this invention, consisting of a single-piece symmetrical shaped geometrical body in an essentially X-shaped embodiment in which the diagonals from the opposite legs cross each other, gives the head support a high structural strength and prevents safely the lateral tilting of the head of the user. This is especially valid if the geometrical body is made from foam material, preferably polyurethane (PUR)-foam. Especially due to its single-piece design the head support can be manufactured easily and economically. At the same time this single-piece design creates other advantages for the user. The head support is lightweight and easy to handle by the user. The head support can be put on and off easily. Most importantly its X-shaped embodiment and its concave geometry of the four outer faces connecting the four legs serve for various different application and positioning options of the head support and increased comfort which will be explained in closer detail in the following.

The basic idea of an X-shaped head support according to this invention allows several advantageous types of embodiments. The four legs of the head support can be of identical length. If the diagonals of the four legs do not cross perpendicularly to each other, a central piece is obtained with rectangular but not quadratic contour. This creates the ability to adapt the head support device to the body of the user by rotation of 90° thus creating two different “support heights”. This is due to the fact that the distance between the legs, or the ends of the legs respectively, which form the chin support bearing or the chest support bearing is smaller or bigger than the distance between the legs, or the ends of the legs respectively, which connect the adjacent legs of the chin support bearing and the chest support bearing. These different distances can be for example 100 millimeters (mm) and 110 millimeters (mm). According to seating position or requirements the user can, by simple rotation of 90°, put on the head support device easily in two different positions to his chin and chest area. Furthermore, the head support is suitable for different persons of different body tallness and different distances between their chins and chests. If this distance is bigger, the respective person can adjust the head support such that the required head tilt angle can be realized according to the respective leg distance.

In a preferred embodiment of the novel head support, where the diagonals formed by the four legs do not cross each other perpendicularly and the four legs have the identical length, it is foreseen that the angles formed by the adjacent legs of the chin support bearing and the chest support bearing are ranging between 70° and 85°. Such range for these angles allows for suitable and sufficient distances of the legs during practical use.

A type of embodiment of the novel head support according to this invention where the diagonals formed by the four legs do cross each other perpendicularly can be advantageous in practical usage as well, even if in this embodiment the different distances between the leg areas as described above are not to be materialized. The X-shaped embodiment of the geometrical body with perpendicular crossing of the leg-formed diagonals still allows various applications. This refers especially to the length and gauge of the concave outer faces. Depending on the chosen length of the legs of the head support different options for the geometrical design are to be materialized which can accommodate for example different shapes and sizes of chins.

An ergonomically specifically beneficial design of the X-shaped head support is created when the outer faces of the free ends of the legs are having a rounded shape. As the geometrical body has a certain thickness, which preferably ranges between one centimeter (cm) and five centimeters (cm), as well as through the rounded leg ends, specifically advantageous features are created for the head support. This enables the application of the head support not only for different head tilt angles, but also for different lateral head positions and lateral tilting angles as well as for different chin and neck shapes of the user. Another important advantage of these ergonomically optimized rounded shapes of the free ends of the legs is to be seen in the fact, that the pressure load at the contact points at the chin and the chest of the user is less pronounced. Hence uncomfortable bruise perception in the area of the chin and the chest is prevented.

Equally advantageous with respect to the ergonomic benefit of the head support according to the invention is its embodiment if the concave outer faces connecting two adjacent legs feature a rounded shape. This is especially valid for the concave connection face between the two legs forming the chin support. By this design the chin and the adjacent neck area of the user can lay comfortably on the support.

As described above, the manufacturing of the novel head support from foam material (especially from polyurethane (PUR)-foam) is beneficial. In another way, the head support can also be made as a hollow or inflatable geometrical body. In the version of an inflatable geometrical body the head support comprises a respective air-inflation valve which does not need further explanation here as those valves are known from neck roll pillows and comparable products already. The advantage of an inflatable head support is that it can be stored on travels with little space requirement. In any case the novel head rest according to this invention is characterized by not being made from any deformable filling material. Such filling material would be counterproductive to the intended function of the head rest because it would reduce the shape stability of the head support. The shape stability however is crucial for the function of the head support.

It was mentioned already that there is a neck strap belonging to the head support. In case of the mentioned head support in its embodiment as a single-piece symmetrical shaped geometrical body of essentially X-shaped geometry, the means to connect the neck strap to the head support comprises a mandrel which is coming out of the center of the middle-piece of the head support. The mandrel is equipped with an X-shaped head and the neck strap is equipped at one of its ends with a buckle holding an aperture which is also X-shaped and allows the introduction of the mandrel with its head into the aperture and the subsequent twisting and sliding of the mandrel within the aperture. At both ends of the neck strap there are means to fix and connect the buckle. In this way the geometrical body is connected to the neck strap and the mandrel which is sliding inside the corresponding aperture of the buckle, allows easy adjustment and rotation of the geometrical body in the area of the chin and the chest of the user. This is especially advantageous if the diagonals formed by the four legs do not cross each other perpendicularly and the distances between the legs are not identically long and consequently, as described above, two different settings or orientations of the head support are possible and two different “support heights” can be achieved. The user can consequently loosen the neck strap a little bit and rotate the head support by 90° around the mandrel into the desired position. In this context it is important to point out that the specific shape of the head support body according to this invention with the four legs and the connecting outer faces in between them creates the advantage that the neck strap runs along the lateral side faces between the chin-support forming and the chest support forming legs when worn by the user. Through the concave shape of these outer faces the neck strap is guided and held in place against sliding over the upper and lower legs. Hence the special shape of the geometrical body provides a good guidance of the neck strap towards the neck of the user and consequently a stable positioning of the head support device at his body.

The X-shaped embodiment of the mandrel head as described above as well as the aperture in the buckle allows rotation and sliding of the mandrel inside the X-shaped aperture of the buckle after the mandrel with its head has been introduced into the aperture and thus the complete geometrical body of the head support can be rotated and slid relative to the buckle. This is possible because the gauges of the mandrel head are a little bit smaller than the gauges of the aperture inside the buckle which enables introduction of the mandrel head into the buckle and release out of it reversely. After introduction of the X-shaped mandrel head into the X-shaped buckle aperture, movement in four directions are possible corresponding to the X-shape of the aperture. In this way, the mandrel with its head can be slid into the four aperture parts of the X-shaped aperture. As the connection of the geometrical body (with the mandrel and its head being attached to it) to the buckle forms a loose connection, the mandrel with its head cannot only be slid sidewards in the aperture of the buckle but it can also be rotated along the axis of the mandrel. This motion option is also possible in the reverse direction, i.e. the mandrel with its head and the buckle can be slid and rotated against each other by the person using or wearing the head support.

This design solution thus enables a simple additional height adjustment at the head or the neck of the user respectively and at the same time an adaptation to the individually most comfortable angular and tilt position of the head support. This adaptability is highly advantageous because—as described before—the tallness of the persons wearing such head support or the distance between their chins and chests is commonly varying quite much. Predominantly because of the varying neck lengths of the users, the shape of the mandrel, the mandrel head, and the aperture of the buckle, as well as the options for sliding and rotating between buckle and geometrical body of the headrest, bring additional advantage. The height variation can be combined with the height adaptation which was described above, namely the type of embodiment of the geometrical body with different distances between the legs, creating for example a smaller leg distance of 100 mm and a bigger leg distance of 110 mm.

As described above, there are means at the buckle and at the ends of the neck strap which allow the connection of these with each other. Hence the user can put on the head support and, after leading the strap around the neck, fix it with the mentioned means. These means can preferably be usual hook and loop fasteners as those are offered by many suppliers. In such way for example the hook-like strip can be arranged at least in some areas at the front side of the buckle which is pointing away from the neck of the user. The corresponding loop means to close this hook and loop connection can be arranged at the other end of the neck strap. The user then has to only connect this end of the neck strap with the hook means at the buckle in order to connect the neck strap with the head support as a whole.

According to the invention a specifically advantageous embodiment or design for the buckle belonging to the neck strap is suggested. In this type of embodiment, the X-shaped aperture in an area facing away from the user during wearing is equipped with an indentation such that the X-shaped mandrel head does not protrude from the buckle when the mandrel is introduced into the aperture. This special design or embodiment respectively generates an additional benefit for the user of the head support because besides the advantages of the aperture and mandrel as described above, the existence of the indentation in the buckle at the position where the X-shaped aperture is arranged serves the purpose that the X-shaped mandrel head does not protrude from the buckle. It is obvious that this is possible if the thickness or height of the mandrel head is less than or identical to the depth of the indentation in the buckle. This has to be considered when manufacturing the buckle and the mandrel head. The mandrel head then does not protrude out of the indentation of the buckle which allows placing it space-saving in the buckle and without hazard of injury for the user. The indentation area can be design as a circular shape which gives the buckle an attractive look.

For the attachment of the hook and loop means at the outer side of the buckle especially those areas outside and around the indentation are favorable. Hence the indentation and the X-shaped aperture in it are covered by the neck strap when the neck strap is connected to the buckle by connecting the hook means at the outer side of the buckle to the loop means at the other end of the neck strap. This generates an attractive look when wearing the head support.

It was mentioned above that the head support is made as a single geometrical body. For the preferred embodiment with the mandrel and its mandrel head, as also described above, this means that the mandrel and mandrel head can be an integral part of the geometrical body and does not have to be an additional or separately fixed part. The geometrical body forming the head support with its middle part, the four legs and the mandrel with its head can thus be made from one piece, for instance made from foam, which comprises the middle part, the four legs and the mandrel with its head. As an alternative to this, the invention proposes another preferred embodiment of the head support where a core part is integrated into the geometrical body, which is containing an aperture for fitting a lid part, for the purpose of which there is a shaft arranged at the lower side of the lid part in the appropriate shape and size to form connection with the aperture of the core part and where there is a mandrel with a mandrel head arranged at the upper side of the lid part.

In this embodiment the mandrel and the mandrel head hence is not an integral part of a one piece geometrical body, but mandrel and mandrel head are arranged in a lid part which is a solitary part before it gets fixed to the geometrical body. During manufacturing of the head support this lid part gets fixed to the geometrical body by attaching it to the core part inside the geometrical body. This arrangement can be materialized preferably by a non-reversible snap connection. This has the advantage that the lid part cannot be removed by the user after it has been fitted to the head support without damaging the head support. The manufacture of such head support according to this invention generates various advantages. Due to the fact that the mandrel and the mandrel head are not inherent parts of the single geometrical body, the utilization of lid parts with mandrels and mandrel heads of different sizes and shapes allows the creation of several variants of the head support. The geometrical body comprising the middle part and the four legs is hence not fixed to a certain embodiment or design of the mandrel and mandrel head. The core part inside the geometrical body is not visible from the outside of the ready-assembled head support because it is covered by the lid part. The core part is located inside the geometrical body and only at that side, where the lid part is attached to it, which is the side facing away from the users or the user's neck, the core part has an aperture into which the shaft of the lid part is introduced. For this reason, the shaft of the lid part and the aperture of the core part feature geometrical shapes which coincide to form a connection, which can be for example cylindrical shapes.

The lid part described above can be made from hard plastics. It can comprise essentially three elements, namely the mentioned shaft, the mandrel with its mandrel head and the flat, lid-like part in between, where this lid part can be manufactured as one single-piece. In assembled state this lid part covers a central area of the middle part of the geometrical body. The middle part of the geometrical body can therefore comprise an indentation for this purpose. According to the thickness of the lid part this indentation can for instance be 3 mm deep.

For optical reasons this lid part can be colored in a different color (for example orange) than the geometrical body (for example black or blue).

The mentioned lid part with its mandrel and mandrel head as well as the buckle can be made from the identical hard plastic. Also, the core part, which is to be integrated into the geometrical body can preferably be made from hard plastic. This way the geometrical body attains additional stiffness as the core part forms the core of the geometrical body. During manufacturing of the head support with core part, the foam is put on around the core part from hard plastics to achieve the specific X-shaped geometrical body. Accordingly, with a head support made out of foam and containing a core part from hard plastics, the outer layers or the shell respectively are relatively soft whereas the core, which is made from hard plastic, is relatively hard. In total this gives the head support a relatively high structural stiffness and at the same time a soft outer shell which increases comfort during utilization. This is specifically valid when using a PUR-type of foam because these types of foam are reversible deformable. They are being manufactured from polyole and isocyanate with a propellant. They are characterized by an open and homogeneous cell structure and the respective permeability for air. The various characteristics, the ease of handling and especially the ease of foaming create advantages also for this specific application. As such the manufacturing process for products from PUR foam does not use substances which consist of monomers, free TDI (toluylendiisocyanate), formaldehydes or asbestos. According to today's knowledge the contact with PUR foam does not trigger any allergic reactions on human skin. Further advantageous characteristics of PUR foams are: good point elasticity, good rebounce-factor homogenous cell structure, good heat- and moisture conductivity, high air permeability, little material fatigue as well as anti-allergic and anti-bacterial properties. Finally, it is to be stated that a head support made from PUR foam is non-toxic and can be manufactured without environmentally harmful ingredients and processes. That is why a head support made from PUR foam is advantageous with respect to health and hygiene. As an alternative to PUR foam also latex can be used.

For the sake of completeness, it shall be stated, that after the embodiment of the head support with the X-shaped mandrel head according to the invention as has been described above, such mandrel head can essentially be designed in any other geometry as well as the aperture of the buckle can be designed in essentially any other shape. This comprises the option for the neck strap to contain at one of its ends a buckle with an aperture of long-hole shape corresponding to a rod-shaped mandrel head allowing the introduction into the aperture as well as the rotation and the sliding inside this aperture. The rod-shaped mandrel head also allows for the height adjustment of the buckle or the head support respectively as described above for the X-shaped mandrel head. Only the sideways adjustment by sliding is not possible as the mandrel can only be slid along the slot line.

It is to be mentioned that the aperture for the introduction of the mandrel does not necessarily need to be integrated into buckle belonging to the neck strap. The fixation of the neck strap to the geometrical body according to this invention can also be done by creation of a respective aperture, for instance of X-shape, in the end of the neck strap itself. It has been found out in tests however, that the use of a buckle with the respective aperture for the mandrel and mandrel head leads to an easier and more safe handling of the head support. The buckle is attached to the respective end of the neck strap preferably with an eyelet in the buckle. This allows a fast replacement of the neck strap or the buckle if required. The neck strap consists preferably of a textile fabric.

Finally, a preferred variant of the head support is referred to, in which a core part of a specifically advantageous shape is integrated into the geometrical body. This core part is a single-piece, symmetrical and essentially X-shaped geometrical body, which comprises a central middle part and four legs which extend outward each diagonally opposite to each other. This core part sits inside the central piece of the geometrical body of the head support with its four legs such that perpendicular to the plane which is spanned by the four legs and out of the center of the core part a connecting element is rising which forms the aperture for fixing the lid part to it or to receive the shaft of this lid part.

This specially shaped core part effectively serves as a reinforcement core for the geometrical body of the head support. Consequently, it has a similar geometrical shape as the geometrical body. Identically, the core part and the geometrical body have a central middle part, from which four legs extend diagonally opposite each other. The four legs of the core part are thus supposed to support or stabilize its four legs inside the geometrical body. At the same time, the core part contains the aperture for attaching the cover part or for receiving the shaft of the lid part.

The invention will be explained in more detail below with reference to the schematic drawings. They show:

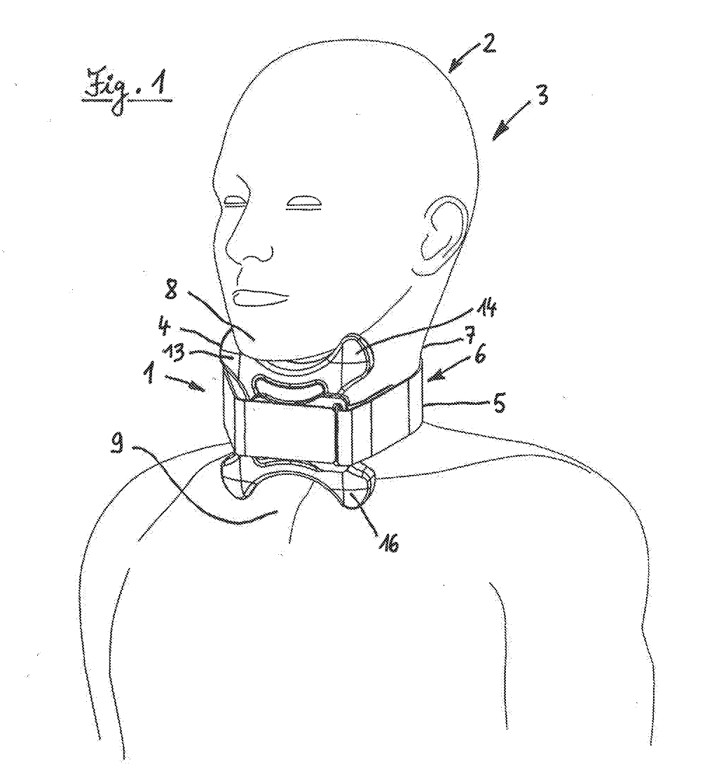

FIG. 1 a perspective view from the front of a first type of embodiment of a head support according to the invention, as it is applied to the neck of a person wearing this head support,

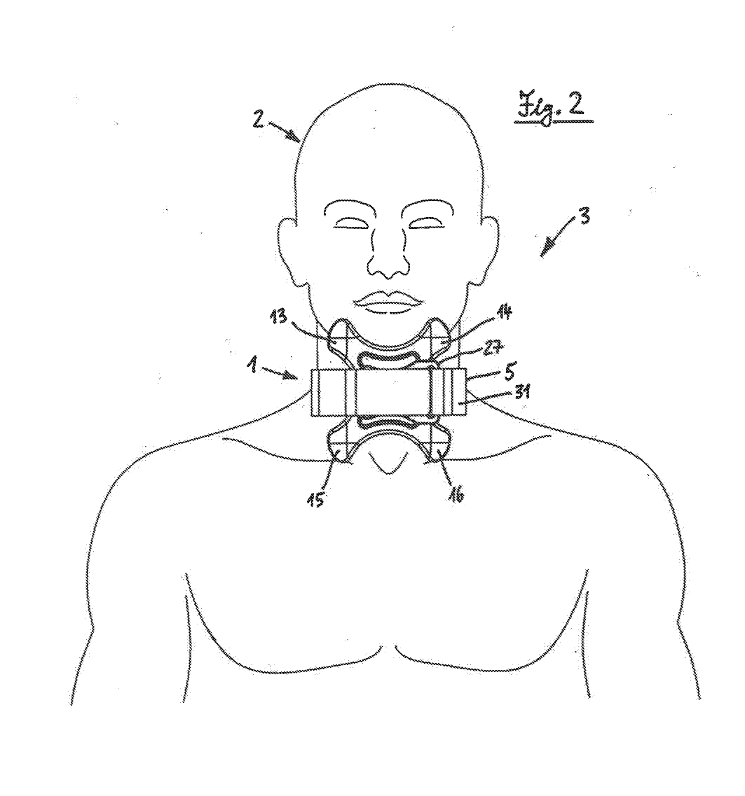

FIG. 2 a view from the front of the person shown in FIG. 1 and with the head support attached to it,

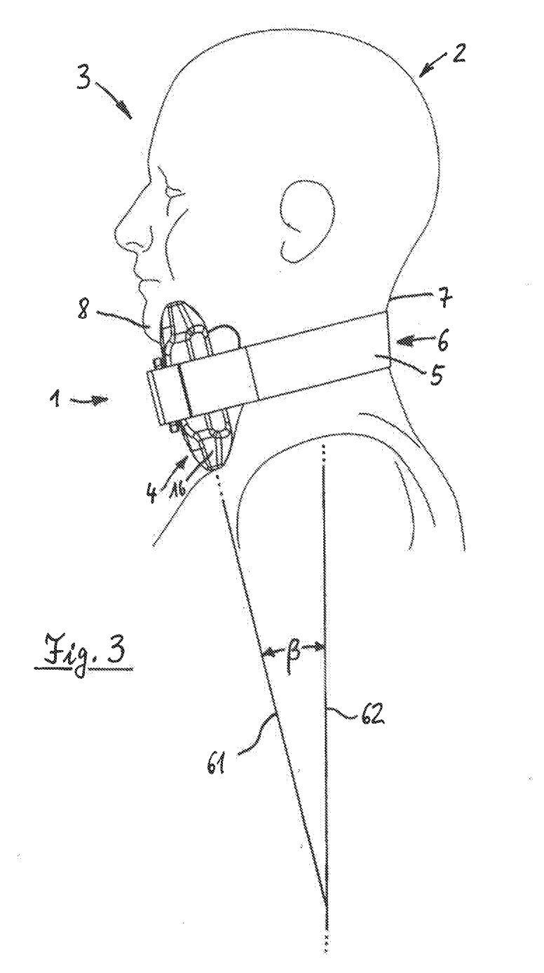

FIG. 3 a view from the side of the person shown in FIG. 1 and FIG. 2 with the head support attached to their necks,

FIG. 4 a view from the side of the geometrical body belonging to the head supports as shown in FIGS. 1-3 with the X-shaped holding mandrel head attached to it,

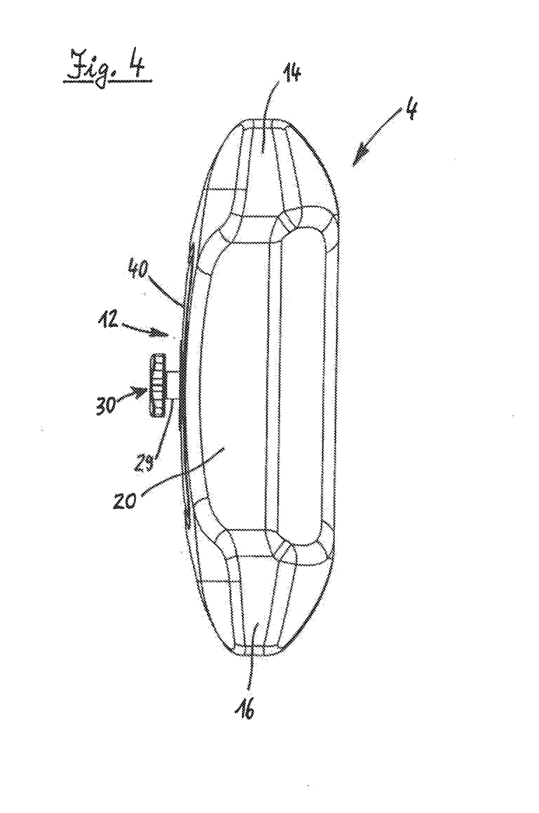

FIG. 5 a top view of the front side of the geometrical body as shown in FIG. 4 where the X-shaped holding mandrel head is located,

FIG. 6 a sectional view of the geometrical body as shown in FIG. 4 and FIG. 5 along the cut line A-A of FIG. 5,

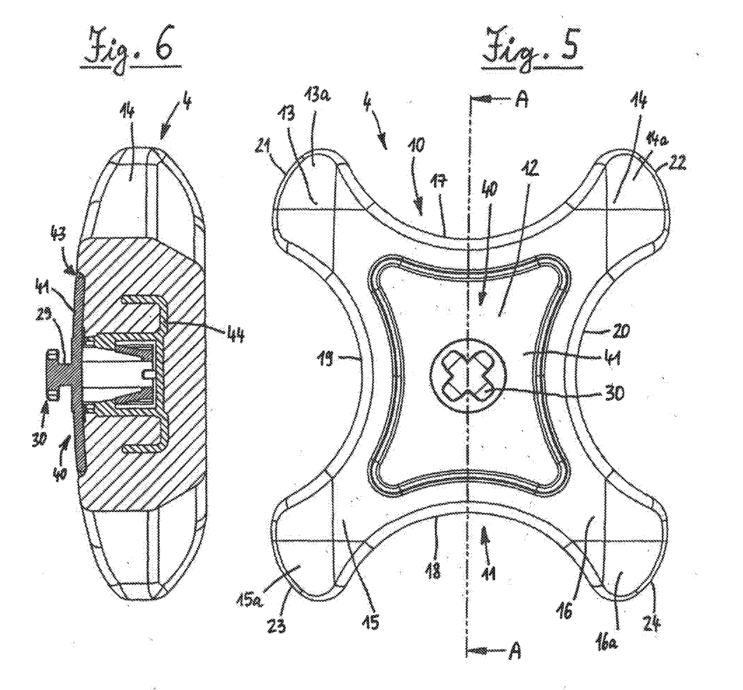

FIG. 7 a top view according to the view as given in FIG. 5 of the outer side of the geometrical body with holding mandrel head indicating the cut line B-B and both of the leg diagonals,

FIG. 8 a sectional view of the geometrical body along the cut line B-B of FIG. 7,

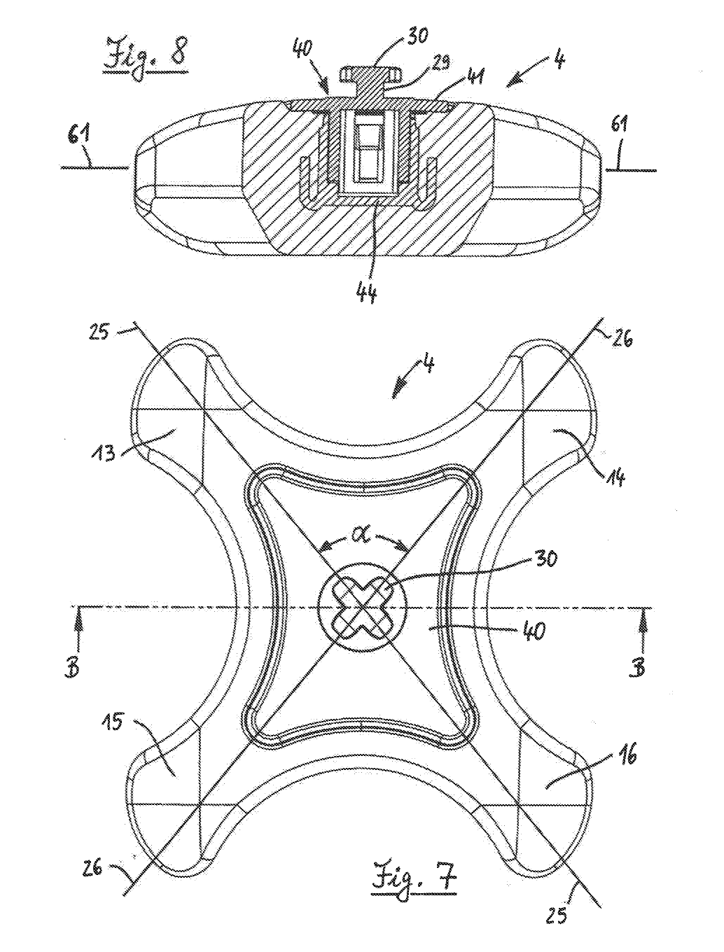

FIG. 9 a view of a type of embodiment of a neck strap belonging to the head support with buckle showing the side of the neck strap or the buckle respectively which contains the recess with the X-shaped aperture,

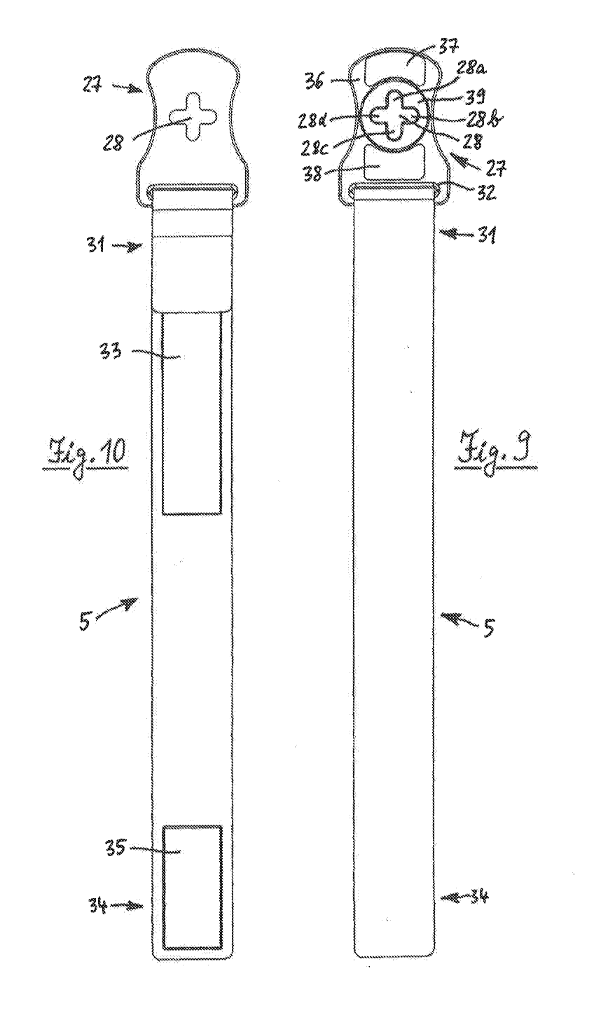

FIG. 10 a view of the other side of the neck strap or buckle respectively as shown in FIG. 9,

FIG. 11 a view corresponding to FIG. 5 on the front side of the geometrical body of the head support as shown in FIG. 1-3 with the neck strap attached to it,

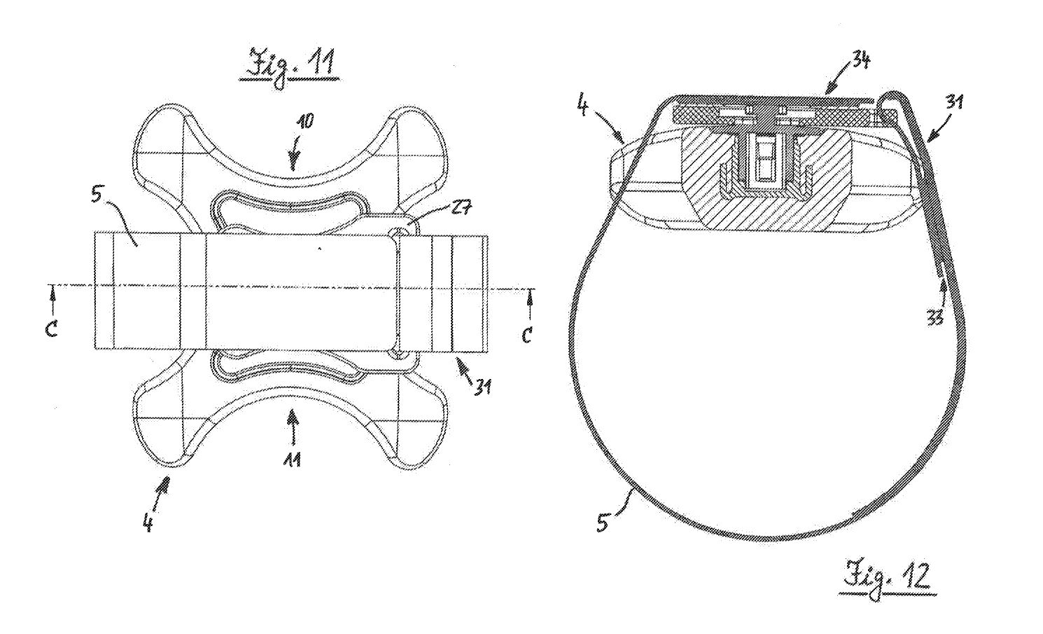

FIG. 12 a sectional view of the head support of FIG. 11 along the cut line C-C of FIG. 11,

FIG. 13 a view corresponding to FIG. 11 on the head support with neck strap where a state is displayed in which the other end of the neck strap is not fixed to the buckle,

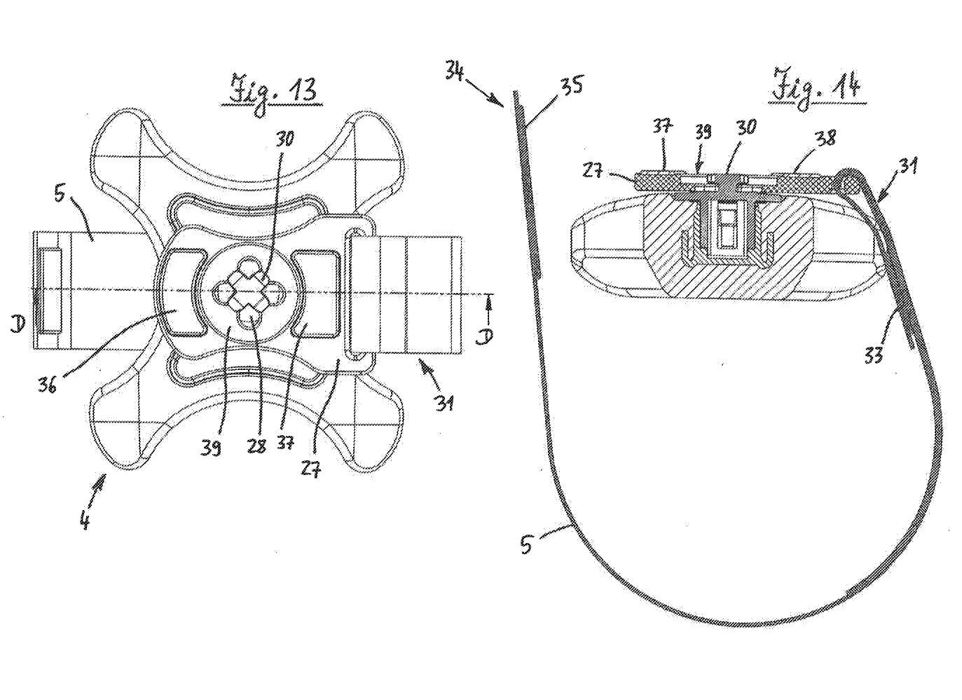

FIG. 14 a sectional view of the head support along the cut line D-D of FIG. 13,

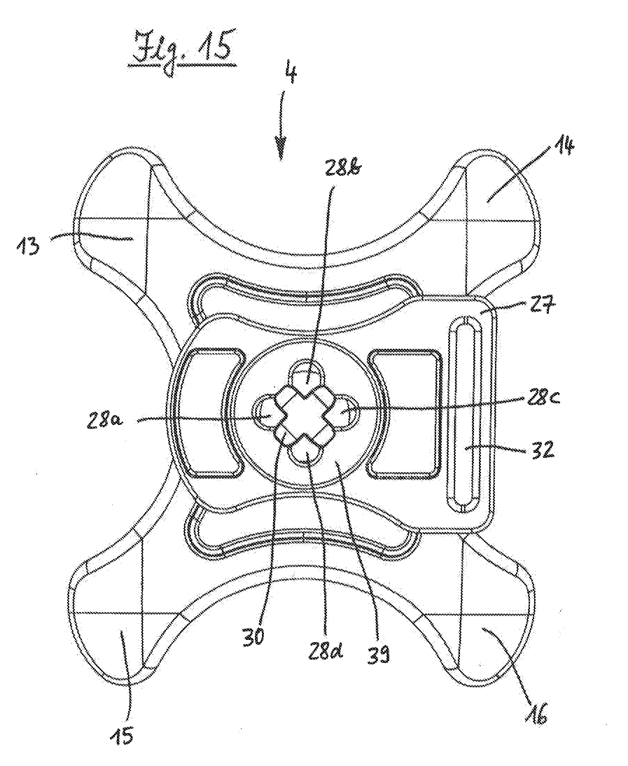

FIG. 15 a view corresponding to FIGS. 5 and 7 of the geometrical body of the head support, where only the buckle attached to the geometrical body is shown and where the X-shaped holding mandrel head is located exactly in the middle of the aperture of the buckle,

FIG. 16 a view corresponding to FIG. 15 where the situation is displayed when the X-shaped holding mandrel head is located in the left aperture part shown on FIG. 16,

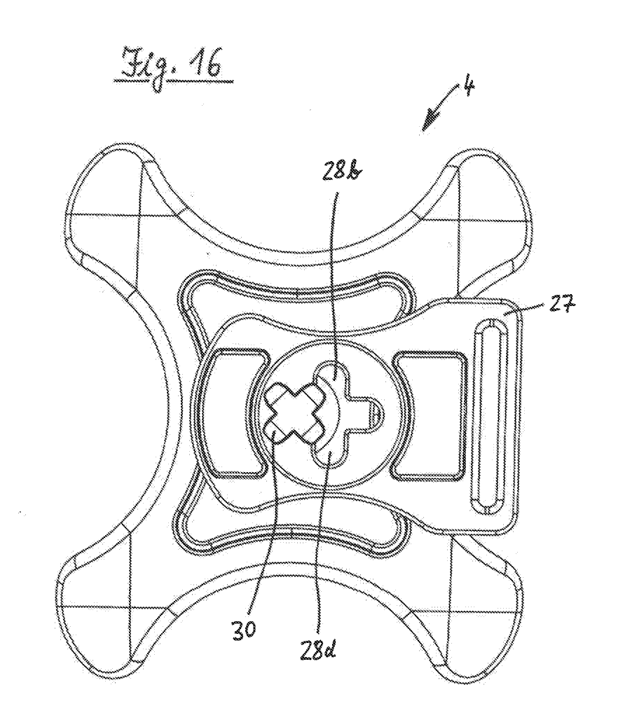

FIG. 17 a view corresponding to FIG. 15 or FIG. 16 where the situation is displayed when the X-shaped holding mandrel head is located in the upper aperture part shown on FIG. 16,

FIG. 18 a view corresponding to FIG. 15, FIG. 16 or FIG. 17 where the situation is displayed when the X-shaped holding mandrel head is located in the lower aperture part shown on FIG. 16,

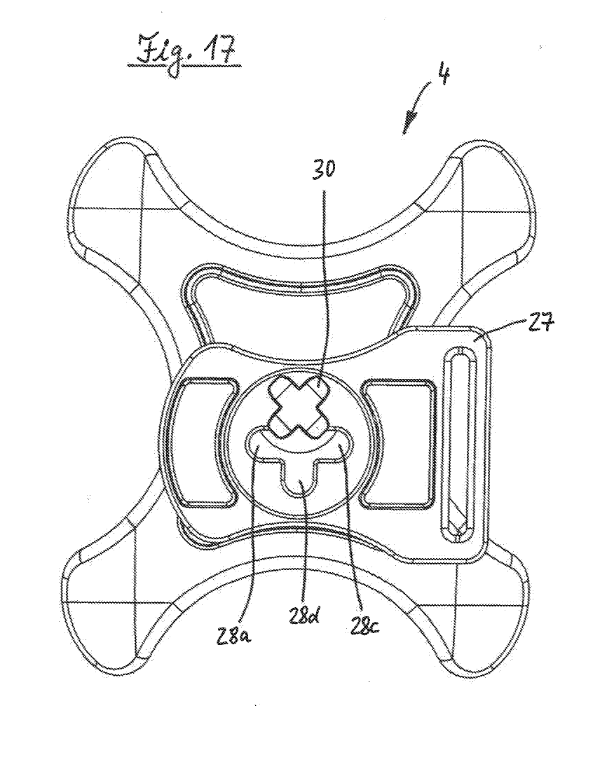

FIG. 19 a view corresponding to FIG. 15 of the geometrical body with the attached buckle, where the geometrical body is in a position rotated by 90° compared to the position in FIG. 15,

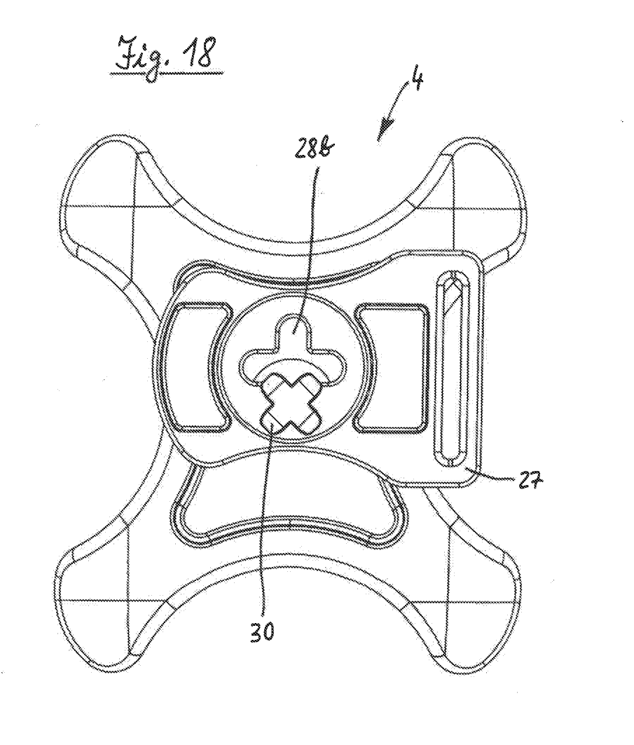

FIG. 20 a top view on the outer side of an embodiment according to the invention of the lid part for the use with the geometrical body of the head support,

FIG. 21 a view on the short side of the lid part of FIG. 20,

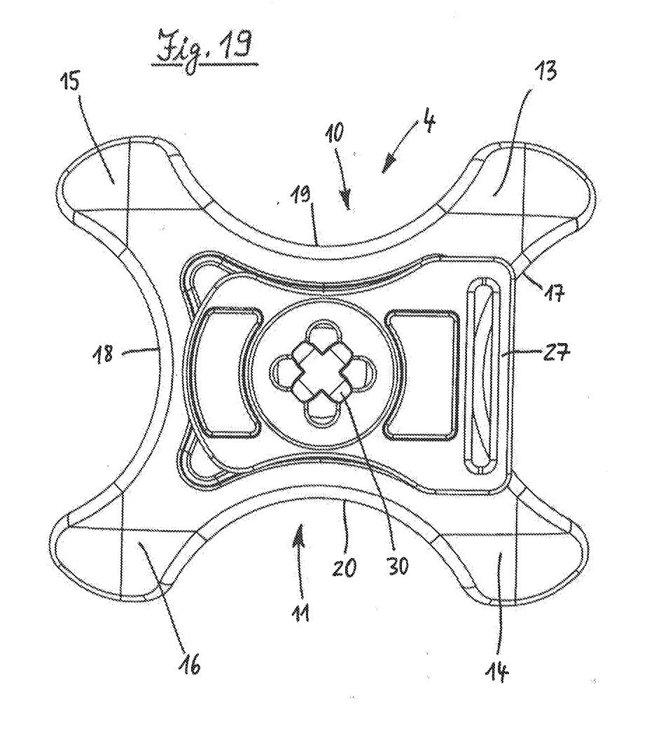

FIG. 22 a view on the long side of the lid part of FIG. 20,

FIG. 23 a view corresponding to the view of FIG. 20 with the cut lines E-E and F-F,

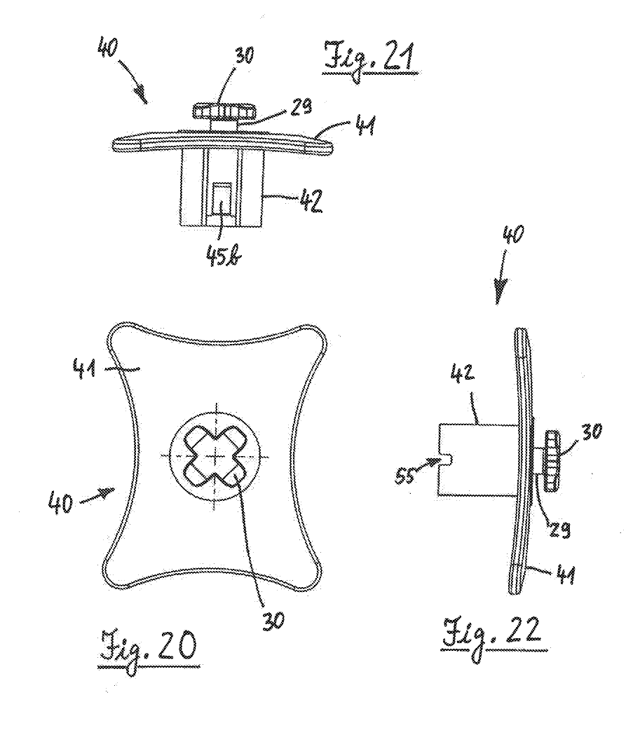

FIG. 24 a sectional view of the lid part of FIG. 23 along the cut line F-F in FIG. 23,

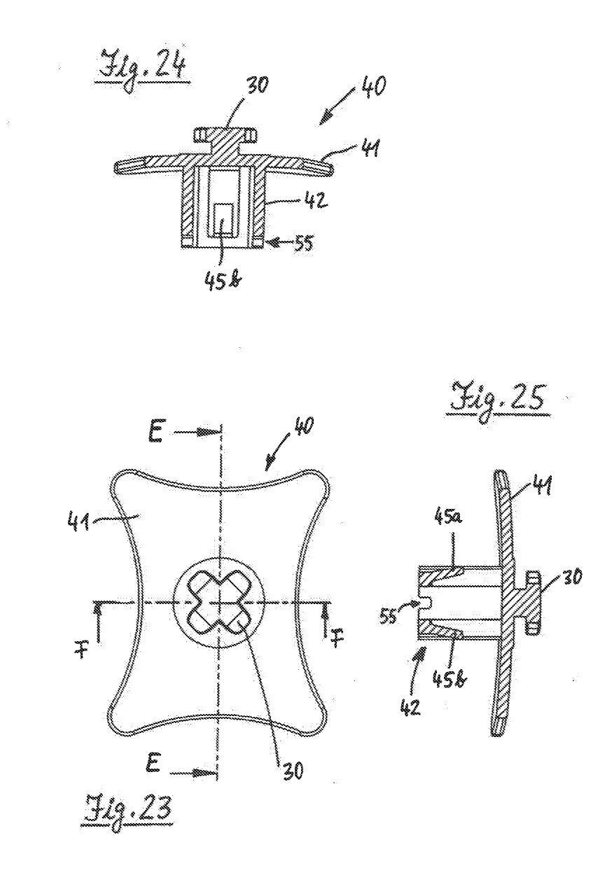

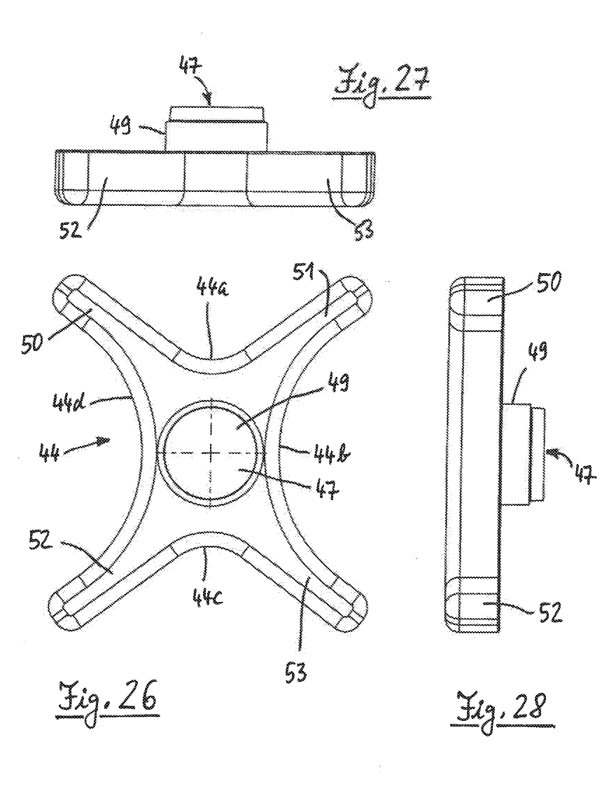

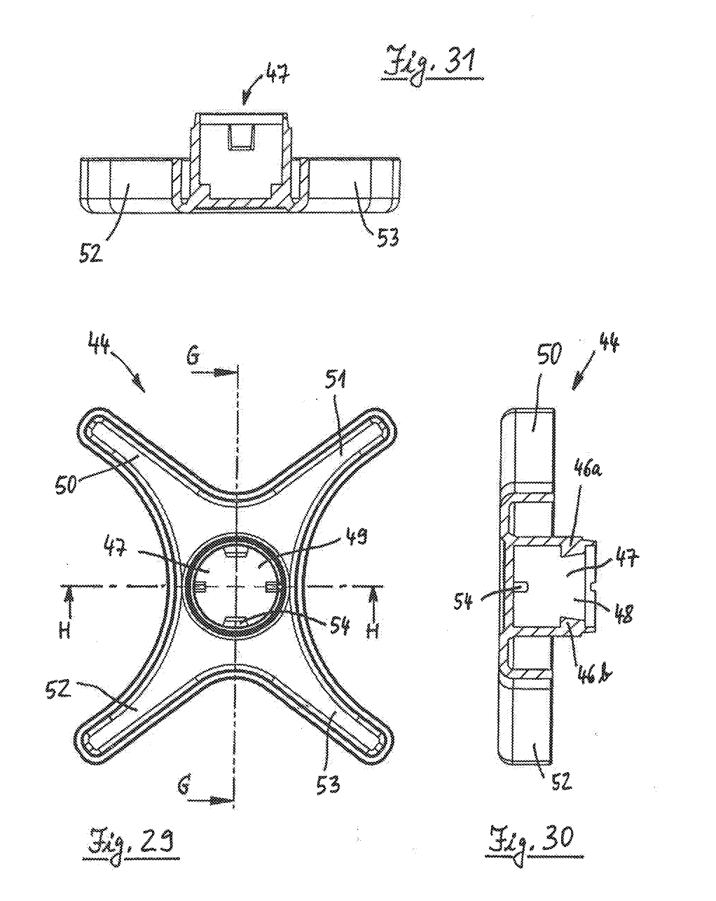

FIG. 25 a sectional view of the lid part of FIG. 25 along the cut line E-E of FIG. 23,

FIG. 26 a top view on one side of the embodiment according to the invention of the lid part for the use in a geometrical body of the head support,

FIG. 27 a view on a short side of the lid part shown in FIG. 26,

FIG. 28 a view on a long side of the lid part shown in FIG. 26,

FIG. 29 a top view on the other side of the lid part shown in FIG. 26 with the cut lines G-G and H-H,

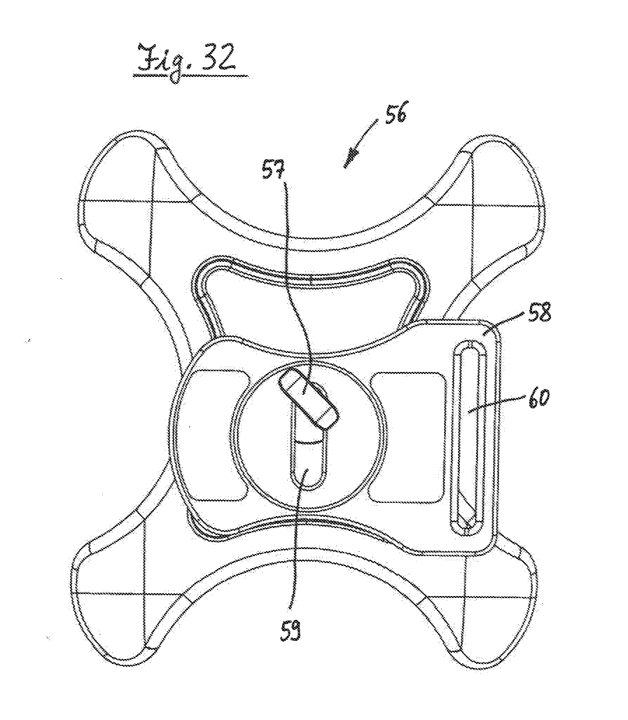

FIG. 30 a sectional view of the lid part of FIG. 29 along the cut lines G-G of FIG. 29,

FIG. 31 a sectional view of the lid part of FIG. 29 along the cut line H-H of FIG. 29,

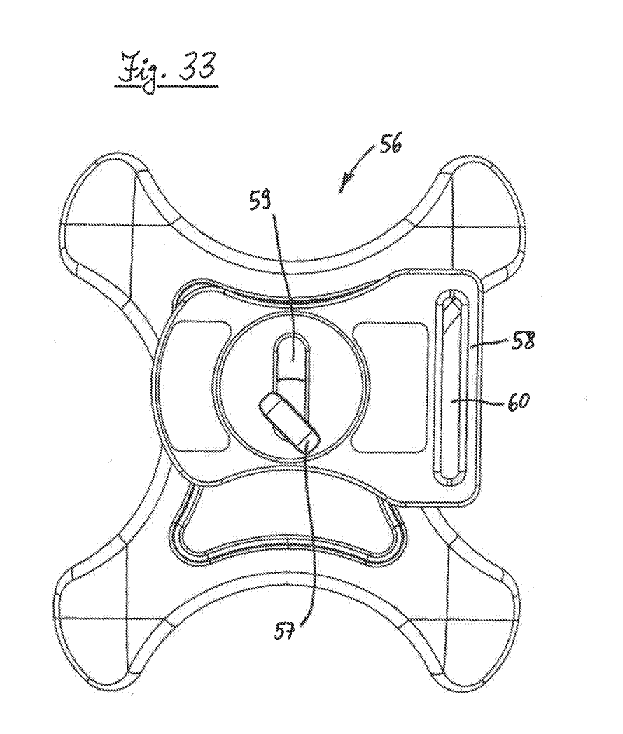

FIG. 32 a view corresponding to FIG. 17 on a further type of embodiment of a head support corresponding to the invention with a geometrical body, to which a rod-shaped mandrel head is joined instead of a X-shaped mandrel head, where the state is shown, in which the mandrel head is located in the upper aperture part of the slot-shaped aperture of the buckle as shown in FIG. 32, and

FIG. 33 a view corresponding to FIG. 18 on the geometrical body, where the state is shown, in which the mandrel head is located in the lower aperture part of the slot-shaped aperture of the buckle of FIG. 33.

FIGS. 1, 2 and 3 show a head support 1 which serves the purpose of supporting the head 2 of a human being or a person 3 respectively. The two main components of the head support 1 are a single-piece symmetrical shaped geometrical body 4, consisting of an X-shaped embodiment, as well as a neck strap 5. FIGS. 1-3 show that the neck strap 5 is led around the neck 7 of the person 3 and holds the head support 1 in its position between the chin 8 and the upper chest area of the person 3. The person 3 is displayed in FIG. 1-3 in an upright position of the body as while standing or being seated. In such position of the body, the head support 1 can be used in order to support the head 2 in the direction towards the chest or the upper chest area 9.

Accordingly, the geometrical body is designed such that it creates a chin support bearing 10 and a chest support bearing 11. The chin support bearing 10 supports the chin 8, while the chest support bearing 11, opposite to the chin support bearing 10, supports at the upper chest area 9. In order to explain this in further detail, the embodiment and shape of the geometrical body 4 is described in the following, also with reference to the FIG. 4 through 8. FIG. 4 displays the geometrical body from a side view corresponding to the view in FIG. 3. FIGS. 5 through 8 are displaying other views on the geometrical body 4, partially in sectional views which are self-explanatory to a large extent. The geometrical body 4 comprises a centrally located middle part 12. This middle part is hence located in the center of the geometrical body 4. Four legs 13, 14, 15 and 16 now extend from this middle part 12. Since it is a symmetrical shaped geometrical body 4, there are two legs diagonally opposite each other, namely legs 13 and 16 on the one side and legs 14 and 15 on the other side. The two adjacent legs 13 and 14 now form, together with the connecting and concave outer surface 17 of the central part 12, the chin support bearing 10. Both the legs 15 and 16, which are opposite to the chin support bearing 10, together with the concave outer surface 18 of the middle part 12, form the chin support bearing 11. In addition, the concavely designed outer surface 19 stretches between the adjacent legs 13 and 15 of the chin support bearing 10 and the chest support bearing 11. Opposite to this outer surface 19 lies the likewise concavely designed outer surface 20 between the legs 14 and 16. For reasons of better ergonomics, the concave outer surfaces 17-20 feature a rounded shape. This is especially visible in FIGS. 4 and 5. The same applies to the free end portions 13a, 14a, 15a and 16a of legs 13-16, the outer surfaces 21, 22, 23 and 24 of which also have a rounded shape. It should be noted that the geometrical body 4 is made of a poly-urethane foam (PUR) material.

By utilization of such material the geometrical body with a variety of shapes which also include curves can be manufactured very well and inexpensively with known manufacturing processes.

The geometry of the geometrical body 4 will be described in more detail in the following. In FIG. 5 it can be seen that the geometrical body 4 with the middle part 12 and the four legs 13-16 forms an X-shape and the respective diagonally opposite legs 13 and 16 or 14 and 15 respectively, form diagonals 25 and 26, which intersect at the center of the middle part 12. These diagonals 25 and 26 are shown in FIG. 7, but omitted in FIG. 5. Thus, it can be seen in FIG. 7 that the diagonals 25 and 26 formed by the four legs 13-16 are of equal length and do not run perpendicularly to one another. Rather, the angle a between the two legs 13 and 14, which form the chin support bearing 10, is less than 90°. In the drawing of FIG. 7, the angle a is approximately 77°. This also means that the outer circumference of the geometrical body 4 formed by the four legs 13-16 is not square-shaped but rectangular. In turn this means that the distance between the free end portions 13a and 14a of leg 13 and 14 and between the free end portions 15a and 16a of leg 15 and 16 is less than the distance between the free end portions 13a and 15a and between the free end portions 14a and 16a. It follows that the geometrical body 4, when it is worn by the person 3 in that position as shown in FIGS. 1-3 and, accordingly, in FIGS. 5 and 7, generates a greater distance between the chin 8 and the upper chest area 9. The head 2 is thus supported in a higher position. Now the geometry of the geometrical body 4 opens up the possibility of setting a different distance between the chin 8 and the upper chest area 9 of the person 3 when wearing the head support 1 by rotating it by 90°. Thus, if the person 3 disengages the neck strap 5, it can rotate the geometrical body from the position shown in FIGS. 1-3 or 5 and 7 by 90° to the left or right and then attach it back to the neck 7. Then no longer the legs 13 and 14 with the associated outer surface 17 (or the opposing legs 15 and 16 with the associated outer surface 18) form the chin support bearing 10 or chest support bearing 11, but the chin support bearing 10 is formed in this case from the legs 13 and 15 with the outer surface 19 and the chest support bearing 11 is formed by the legs 14 and 16 with the outer surface 20 (or vice versa). Thus, by rotating the X-shaped geometrical body 4 by 90°, an adaptation of the same on the body of the person 3 to two different “heights” is possible. This is one of the decisive advantages of the head support 1 according to the invention.

It is clear that the dimensions or distances of the legs 13-16 are completely freely selectable. Their selection depends primarily on the different body sizes and proportions of humans. In other words, larger geometrical bodies 4 are suitable for taller people or people with longer necks, while for smaller people accordingly smaller geometrical bodies 4 are useful. In order to be able to take into account the different body sizes and proportions, head supports according to the invention can be offered with a large number of differently sized geometrical bodies. By way of example, a geometrical body 4 is given in which the distance between the free end regions 13a and 14a of legs 13 and 14 is approximately 100 mm, while the distance between the free end regions 13a and 15a of legs 13 and 15 may be 110 mm, for example. The difference of these distances should not be too low, because otherwise when wearing the head support 1 only two very similar “heights” can be made possible. By the design of the geometrical body 4 with different distances or heights, it is also possible that the same head support 1 can also be used well by two persons who are of different sizes or have different body proportions, for example different distances between her head or chin and her chest area. For example, the taller person may rather wear the head support to be “higher”, meaning to form a greater distance between the chin 8 and the upper chest area 9. In this respect, however, there are basically no restrictions on the use of head support by various people.

Wearing the head support 1 with the geometrical body 4 is comfortable because of the concave and rounded outer surfaces 17-20 and the rounded outer surfaces 21-24 at the free end portions 13a, 14a, 15a, 16a of the legs 13-16. These ergonomically favorable shapes and curves allow the head support 1 to be used not only in different tilt angles of the head 1 but also in different side/tilt positions of the head support for adaptation to positions felt individually comfortable by the user as well as for different neck and chin shapes of the person 3. In connection with the use of the already mentioned poly-urethane foam material (PUR), the advantages of which have already been described in detail above, the use of the head support 1 also leads to a low compressive load when resting the head support 1 on the chin 8 and at the upper chest area 9, so that unpleasant pressure points are avoided there.

In the following it is explained how the geometrical body 4 is connected to the neck strap 5, which serves to attach the head support 1 to the person 3, and what additional advantages result therefrom. Reference is first made to FIGS. 9 and 10, in which a type of embodiment of neck strap 5 can be seen. To this neck strap 5 belongs a buckle 27. This buckle 27 has an X-shaped aperture 28, which in turn-due to the X-shape—features four apertures partial areas 28a, 28b, 28c and 28d. This aperture 28 serves to introduce a holding mandrel 29 attached to the center part 12 of the geometrical body 4 and projecting therefrom, as well as a holding mandrel head 30 arranged thereon and also having an X-shaped embodiment, which can well be seen in FIGS. 4-8.

FIGS. 11, 12, 13 and 14 show how the neck strap 5 and the attached buckle 27 respectively is connected to the geometrical body 4, through person 3 introducing the retaining pin 29 and the holding mandrel head 30 arranged in it into the aperture 28 of the buckle 27. This state can be seen for example in FIG. 13 and the associated sectional view of FIG. 14. It can also be seen how the neck strap 5, on which the buckle 27 is mounted, runs.

The attachment of the buckle 27 at one end 31 of the neck strap 5 takes place here by means of an eyelet 32, prepared here in the hard plastic, of the buckle 27. This eyelet 32 is also clearly visible in FIGS. 9 and 10 and in FIG. 15. It has already been said that the neck strap can be made from a textile fabric material that is waterproof and tear resistant. Such bands are already known, for example, from bags. The neck strap 5 is detachably connected to the buckle 27 by means of hook and loop fastening means which are located at the end 31 of the neck strap and form a strip-shaped hook-and-loop closure region 33. This is to be seen in FIG. 10 and also in

FIGS. 12 and 14, the hook-and-loop fastener region 33 is easy to see. At this point it should be mentioned that the hook and loop portion 33 is designed in a way that the neck strap 5 can be adjusted in its length so that it can be used by different people easily.

Also, the other end 34 of the neck strap 5 has a strip-shaped hook and loop closure region 35, on the side of the neck strap 5 shown in FIGS. 10 and 14. For applying the head support 1 or for attaching the end 34 of the neck strap 5 with the hook-and-loop fastener region 35, two, by the way slightly recessed, hook and loop fastener middle regions 37 and 38 opposite the outside 36, are provided on the outside 36 of the buckle 27 and laterally next to their aperture 28. In FIG. 9, the shape of these hook and loop fastener regions 37 and 38 and their arrangement can be seen only on the one outer side 36 of the buckle. This outer side 36 is, when the head support 1 is worn, the outside of the buckle 27 facing away from the body of the person 3 (see FIG. 14). Thus, the hook and loop portion 35 of the free end 34 of the neck strap 5 comes in the applied state of the head support 1 on the corresponding hook and loop parts that are in the hook and loop closing portions 37 and 38 of the buckle 27 (compare FIG. 12). It can be seen in FIGS. 12, 13 and 14 as well as in FIG. 9, that the buckle 27 has on its outer side 36 a recessed area 39 in which the X-shaped aperture 28 is located. This recessed area 39 has approximately the height corresponding to the height or thickness of the holding mandrel head 30 so that the retaining mandrel head 30, when it is in the X-shaped aperture 28, does not project beyond the outside of the buckle 28.

Thus, if then the end 34 of the neck strap 5 is attached to the outside of the buckle 27, there is no projecting point due to the retaining mandrel head 30. The outwardly facing side of the end 34 of the neck strap 5 of the head support 1 during wearing is thus perfectly smooth or flat. This situation can be clearly seen in FIGS. 12 and 14, respectively.

In this context, it should also be noted that in particular FIG. 1 shows the course of neck strap 5 at the neck 7 and in the region of the geometrical body 4. From FIG. 1 it is also seen that the concave outer surfaces 19 and 20 allow a guide for the neck strap 5 back to the neck 6. The neck strap 5 thus also stabilizes the position of the geometrical body 4 in this manner. The concave outer surfaces 19 and 20 thus also have a lateral guiding function in this case.

The position of the geometrical body 4 shown in FIG. 15 and the buckle 27 attached thereto (belonging to the neck strap 5, not shown here) correspond to the position of this geometrical body 4 shown already shown in FIG. 13. With FIG. 15 and the subsequent FIGS. 16, 17 and 18, the possibilities of adjustability of the geometrical body 4 will now be described which result from the X-shape of the retaining mandrel head 30 and the X-shape of the aperture 28 of the buckle 27. FIGS. 15 to 18 each show four different positions of the retaining mandrel head 30 in the aperture 28. These various positions are possible because the retaining mandrel 29 and the retaining mandrel head 30 attached thereto are displaceable and rotatable in the aperture 28. This also applies in the reverse sense, that is, the aperture 28 of the buckle 27 can also be displaced or rotated relative to the retaining mandrel 29 or the retaining mandrel head 30. This has already been explained above. In addition, these possibilities will be explained with reference to FIGS. 15-18. Thus, FIG. 15 shows the position of the retaining mandrel head 30, as it is located exactly in the center of the aperture 28. In this case, the geometrical body 4 with the retaining mandrel head 30 was rotated by 90° relative to the X-shaped aperture 28, so that in this way the buckle 27 is locked to the geometrical body 4. In this context, it should be mentioned, and this also results from the sectional views according to FIGS. 12 and 14 showing that the height of the recessed area 39 of the buckle 27 corresponds approximately to the height of the holding mandrel head 30, otherwise it would not be possible for the retaining mandrel head 30 to occupy the position shown in FIG. 15 in the aperture 28 or the recessed area 39.

It can now be seen in FIG. 16 that the retaining mandrel 29 or the retaining mandrel head 30 is in a different position, specifically in the aperture part region 28a. This is usually the state or the position in which the geometrical body and the buckle are located when the head support 1 with the neck strap 5 is finished on the person 3.

FIG. 17 shows the possibility that the retaining mandrel 29 or retaining mandrel head 30 respectively is located in the aperture section 28b of the aperture 28. It can be seen in FIG. 17 that the buckle 27 is “lower” in this position. Or conversely, one can say that the geometrical body 4 in this position is “higher” on the body of the person 3. This means that the X-shape of the aperture 28 with the aperture parts 28b (FIG. 17) and 28d (FIG. 18) allows additional height adjustability of the geometrical body 4 and the head support 1 overall. This shaping thus allows the person 3, which carries the head support 1, in addition to the adjustability, as already described above, of the geometrical body 4 by rotation of it by 90° to make available the two “support heights” for further height adjustment. Likewise, this changed position of the fixation point enables a different angular position of the head support 1 between the chin and chest abutment area of the person 3 and thus a further adaptation to the positions of the head support 1 as individually perceived comfortable. The aforementioned angular position refers to the angle β, which is formed between the median plane (61) of the geometrical body 4 (see FIG. 8) and the vertical line 62 of the person 3 to be regarded as an extension of the spinal column. This vertical line 62 already mentioned in the introduction to the description, as well as the aforementioned center plane 61 and the angle β formed by these are shown in FIG. 3. Thus, by changing the fixing point, that is, the position of the retaining pin 29 or retaining mandrel head 30 in the aperture 28 of the buckle 27, a change in the angle β can be effected. These additional functions of the adjustment due to the X-shape of the aperture 28 of the buckle 27 are further decisive advantages of the inventive head support 1. In FIG. 18 it is shown for the sake of completeness how the buckle 27 positioned above the center of the geometrical body 4 by the retaining pin 29 being located with the retaining mandrel head 30 in the aperture portion 28d.

The illustration of the geometrical body 4 according to FIG. 19 serves to further illustrate the possibility of rotating the geometrical body 4 by 90° in order to use a different “height” of the same. The representation of FIG. 19 is therefore to be seen in comparison to the representation according to FIG. 15. The representations of FIGS. 15 and 19 are identical in principle and have only the difference that the position of the geometrical body 4 is rotated in each case by 90°. It has already been said above that the resulting “height adjustability” of the geometrical body 4 can be combined with the height adjustment of the buckle 27 of the connected neck strap 5 as a result of the X-shaped aperture 28.

It can already be seen in FIGS. 6, 8, 12 and 14 that the retaining pin 29 with the retaining mandrel head 30 seated thereon is arranged on a so-called lid part 40. In the sectional views of FIGS. 6 and 8, the shape and the components of this lid part 40 can already be seen. In addition, FIGS. 20 to 25 show once more the exact embodiment of this lid part 40, which is produced as a single piece from a hard plastic. Thus, the lid part 40 comprises the already mentioned retaining mandrel 29 or retaining mandrel head 30, a lid-like element 41 and a cylindrical shaft 42. The lid-like element 41 is located between the retaining pin 29 and the shaft 42. The outer contour of the lid-like element 41 can also be seen in FIG. 7, for example. For optical reasons and reasons of space savings, the lid-like element 41 is arranged in an indentation 43 provided in the middle part 12 of the geometrical body 4.

During manufacturing of the head support 1, the lid part 40 is connected to the geometrical body 4. In the type of embodiment shown, an intractable connection is created by the shaft 42 of the lid part 40 arranged on the underside of the cover-like element 41 being inserted into a core part 44 which is already located in the geometrical body 4 and will be described in more detail below with reference to FIGS. 26-31. In addition, sectional views of the core part 44 can also be taken from FIGS. 6, 8, 12 and 14. Since the lid part 40 and the core part 44 in the finished state of the head support 1 are firmly connected parts, this will now be explained with reference to the above-mentioned various drawings in detail. The geometries of the lid part 40 and the core part 44 are shown in FIGS. 20-25 and 26-31 completely. This also applies to the means for connecting the cover part 40 and the core part 44. In the illustrated form of a permanent connection, the shank 42 of the cover part 40 has two snap-in lugs 45a which make this latching or snap connection possible, and which are slightly flexible due to the plastic material used and the slightly flexible snap-in lugs. Their position in the cylindrical shaft 42 can be seen for example in FIG. 25. Suitably fitting for this shaft 42 and the latching noses 45a and 45b, snapping enabling and protruding latching noses 46a and 46b are provided in an aperture 47 of the core member 44. Their shape and positions are clearly visible in FIG. 30, for example. The aperture 47 is formed by a connecting element 48 of such cylindrical shape of the shaft 42 has a corresponding cylindrical shape and rises out of the middle part 49 of the core part 44.

The core part 44 is a single-piece manufactured, symmetrical and substantially X-shaped member. Starting from its middle part 49, four legs 50, 51, 52 and 53 extend diagonally opposite one another. Their shape can be seen in FIGS. 26 and 29. The connecting element 48 thus rises perpendicular to the plane formed by the legs 50-53. In the production of the head support 1, the previously prepared core part 44 is surrounded by the poly-urethane foam (PUR) geometrical body 4. The geometrical body 4 thus contains the core part 44 and is then provided with the lid part 40 for the product to be ready for sale. This finished state can be clearly seen in the sectional views of FIGS. 6 and 8 as well as 12 and 14. It can also be seen in these sectional views how the respective latching noses 45a and 45b of the cover part 40 cooperate with the latching noses 46a and 46b of the core part by latching. The latching noses 45a and 45b of the cover part 40 thus arrive at the insertion of the shaft 42 behind the latching noses 46a and 46b of the core part 44, so that thereafter the lid part 40 cannot be removed from the core part 44. Incidentally, four projections 54 are provided on the bottom of the connecting element 48 of the core part 44—distributed over the circumference—which point upwards and, when the lid part 40 is joined to the core part 44, come into apertures 55 which are located on the bottom of the shaft 42. The interlocking projections 54 and apertures 55 additionally serve to fix or hold the lid part 40 in the core part 44.

The geometry of the core part 44, which also has four, the legs 51-54 respectively connecting concave outer surfaces 44a, 44b, 44c and 44d, and its arrangement within the geometrical body 4 cause a stabilization of leg 13 to 15, since the core part 44 is made of a harder plastic material, while the foam material of the geometrical body 4 is softer. The resulting advantages in the use of the head support have already been described in detail above.

Finally, with reference to FIGS. 32 and 33, another type of embodiment of the head support with a geometrical body 56 is shown. This geometrical body 56 differs from the geometrical body 4 only in that a differently shaped holding mandrel head 57 is used. This retaining mandrel head 57 is not X-shaped but rod-shaped. An insertion and removal of this retaining mandrel head 57 from the associated buckle 58 (or vice versa) is thereby made possible that this buckle 58 has a slot-shaped aperture 59. This embodiment can be seen in FIGS. 32 and 33, it being noted that otherwise the geometrical body 56 and buckle 57 will look and function the same as the geometrical body 4 with the buckle 27. Thus, to avoid repetition, reference is made to Figs. referenced in the above description. The views according to FIGS. 32 and 33 therefore correspond in principle to the views of FIGS. 17 and 18, which also need not be explained in detail. At the buckle 58 is therefore also an eyelet 60 to attach a neck strap.

1. Head support for supporting the head of a person in an upright position of the body towards the chest of the person wearing the head support, wherein a part of the head support forms a chin support for the chin of the head and part of the head support opposite to the chin support forms a chest support on the upper chest area of the person wearing the head support and the head support includes a neck strap that can be carried around the neck of the person wearing the head support and holds the head support in its position between the chin and the upper chest area of the person wearing the head support,
characterized,
in that the head support (1) is formed as a one-piece symmetrical shaped geometrical body (4, 56) and this geometrical body (4, 56) has a substantially X-shaped embodiment such that it has a central middle part (12) and that from this central middle part extend outwards, in each case diagonally opposite to each other, four legs (13, 14, 15, 16), wherein two adjacent legs (13, 14) together with the concave outer surface (17) connecting these legs (13, 14) form the chin support bearing (10) and the two legs (15, 16) opposite to this chin support bearing (10) together with the concave outer surface (18) connecting these legs (15, 16) form the chest support bearing (11), that the outer surfaces (19, 20) of the central part (12) between two each adjacent legs (13, 15; 14, 16) of the chin support bearings (10) and the chest support bearings (11) are also concave and that at the side facing away from the body of the person wearing the head support (1) when worn, there are means for fixing the neck strap (5) at the outer side of the central part (12) and in the region of the intersection of the diagonals (25,26) formed by each two legs of the four (13, 14, 15, 16).

2. Head support according to claim 1, characterized in that the four legs (13, 14, 15, 16) have the identical length.

3. Head support according to claim 1 or 2, characterized in that the two diagonals (25, 26) formed by the four legs (13, 14, 15, 16) are not perpendicular to each other.

4. Head support according to claim 3, characterized in that the four legs (13, 14, 15, 16) have the identical length and the angle (a) between the both legs (13, 14; 15, 16), which are generating the chin support bearing (10) or the chest support bearing (11) respectively are ranging between 70° and 85°.

5. Head support according to claim 1 or 2, characterized in that the two diagonals formed by the four legs are perpendicular to each other.

6. Head support according to any of the previous claims characterized in that the outer surfaces (21, 22, 23, 24) of the free end pieces (13a, 14a, 15a, 16a) of the four legs (13, 14, 15, 16) feature a rounded shape.

7. Head support according to any of the previous claims characterized in that, the concave outer surfaces (17, 18, 19, 20) connecting each two of the legs (13, 14, 15, 16) have a rounded shape.

8. Head support according to any of the previous claims characterized in that it is manufactured from a foam material.

9. Head support according to any of the claims 1 through 7, characterized in that it consists of a air-filled or inflatable geometrical body (4).

10. Head support according to any of the previous claims characterized in that the means to attach the neck strap (5) to the head support (1) comprise a holding mandrel (29) which is fixed to and protruding from the head supports (1) middle piece, to which an X-shaped mandrel head (30) is fixed, that the neck strap (5) features at one of its ends (31) a buckle (27) with an aperture (28) where this aperture (28) has an X-shaped, and to the shape of the mandrel head (30) corresponding, embodiment enabling the introduction of the mandrel head (30) into the X-shaped aperture (28) of the buckle (27) and a rotation as well as a sliding of the holding mandrel (30) inside the aperture (28) of the buckle (27), and that there are means to attach the other end (34) of the neck strap (5) as well as at the buckle (27) in order to attach this end (34) of the neck strap (5) to the buckle (27).

11. Head support according to any of the previous claims characterized in that the means to attach both of the ends (31, 34) of the neck strap (5) are hook and loop connectors.

12. Head support according to claim 11, characterized in that the X-shaped aperture (28) is located in a recessed area (39) at the outer surface of the buckle (27) which is facing away from the body when the head support (1) is worn, such that the X-shaped mandrel head (30) is not protruding this recessed area (39) when it is sitting inside the X-shaped aperture (28).

13. Head support according to claim 12, characterized in that there are hook and loop connecting means present at the non-recessed part (39) at the outer surface (36) of the buckle (27) allowing to attach the other end (34) of the neck strap (5).

14. Head support according to any of the previous claims, characterized in that there is a core part (44) integrated into the geometrical body (4) which holds an aperture (47) for mounting a lid part (40), for the purpose of which at the bottom side of the lid part (40) there is a shaft (42) and this shaft (42) and the aperture (47) of the core part (44) feature a mutually fitting shape and means to make a connection, and that there is a holding mandrel (29) with a mandrel head (30) at the upper surface of the lip part (40).

15. Head support according to any of the previous claims, characterized in that the geometrical body (4) consists of a foam material and the core part (44), the lid part (40) and the buckle (27) consist of a hard plastic material.

16. Head support according to any of the previous claims 10 through 15, characterized in that the mandrel head (57) on top of the holding mandrel is not X-shaped but rod-shaped, so that the neck strap contains a buckle (58) at one of its ends with an aperture (59) which has a long-hole shape corresponding to the rod-shape of the mandrel head (57) as well as an embodiment allowing the introduction of the mandrel head into the long-hole type aperture (59) of the buckle (58) and its rotation and sliding in the aperture (59) of the buckle (58).

17. Head support according to any of the claim 14, 15 or 16, characterized in that the core part (44) is a single-piece, symmetrical and essentially X-shaped geometrical body which comprises a central part (49) from which extend, each diagonally opposite to each other, four legs (13, 14, 15, 15), and this inside the four legs (50, 51, 52, 53) of the geometrical body (4) of the head support (1), and that a connecting element rises perpendicularly to the plane which is formed by the four legs (50, 51, 52, 53) from the central part (49) of the core part (44) which builds the aperture (47) for mounting the lid part (40) or its shaft (42) respectively.
